(19) United States
(12) Patent Application Publication
(10) Pub. No.: US 2026/0057525 A1
(43) Pub. Date: Feb. 26, 2026

(54) SYSTEMS AND METHODS FOR PERFORMING VESSEL SEGMENTATION FROM FLOW DATA REPRESENTATIVE OF FLOW WITHIN A VESSEL

(71) Applicant: PURDUE RESEARCH FOUNDATION, West Lafayette, IN (US)

(72) Inventors: Pavlos Vlachos, West Lafayette, IN (US); Vitally Rayz, West Lafayette, IN (US); Sean Routhenberger, Sunnyale, CA (US)

(21) Appl. No.: 19/102,579

(22) PCT Filed: Aug. 1, 2023
(86) PCT No.: PCT/US2023/029195

§ 371 Date: Feb. 10, 2025

Related U.S. Application Data
(60) Provisional application No. 63/397,153, filed on Aug. 11, 2022.

(51) Int. Cl. G06T 7/11; A61B 5/02; A61B 5/026; G06T 7/00; G06T 7/73

(52) U.S. Cl. CPC G06T 7/11; A61B 5/02007; A61B 5/0263; G06T 7/0012; G06T 7/73; G06T 2200/04; G06T 2207/10088; G06T 2207/30104

(57) ABSTRACT

The invention generally provides systems and methods for performing vessel segmentation from flow data, such as but not limited to 4D flow Magnetic Resonance Imaging (MRI) data. In certain aspects, the systems and methods of the invention may involve receiving flow data representative of flow in a vessel (such as 4D MRI flow data); identifying net flow effects in the flow data (such as 4D MRI flow data) according to a standardized difference of means (SDM) velocity that involves quantifying a ratio between net flow and observed flow pulsatility in each voxel of the received flow data (such as 4D MRI flow data); and identifying voxels with higher SDM velocity values than stationary tissue voxels, thereby performing vessel segmentation from flow data (such as 4D MRI flow data).

### RELATED APPLICATION

This application claims the benefit of and priority to U.S. provisional patent application Ser. No. 63/397,153, filed Aug. 11, 2022, the content of which is incorporated by reference herein in its entirety.

### GOVERNMENT SUPPORT

This invention was made with government support under NS106696 and HL115267 awarded by the National Institutes of Health. The government has certain rights in the invention.

### FIELD OF THE INVENTION

The invention generally provides systems and methods for performing vessel segmentation from flow data representative of flow within a vessel, such as but not limited to 4D flow Magnetic Resonance Imaging (MRI) data.

### BACKGROUND

4D flow MRI is a phase-contrast magnetic resonance imaging (PC-MRI) technique capable of measuring time-resolved 3D velocity fields. This imaging modality has gained interest in the clinical community for its ability to provide non-invasive measurements of blood flow velocity in vivo, which can be used to assess hemodynamic metrics associated with cardiovascular disease progression. For example, wall shear stress (WSS) is associated with atherogenesis and aneurysm initiation and growth. However, this imaging modality suffers from acquisition and processing-related error sources. It has been demonstrated that the error in 4D flow MRI deteriorates the accuracy of hemodynamic metrics evaluated in cerebral aneurysms. Methods are available to correct for partial volume (PV) effects and bias error in 4D flow MRI; however, these approaches necessitate accurate vessel segmentations. Moreover, other researchers have shown that errors in hemodynamic metrics can be mitigated if provided with reliable vessel segmentations.

Commonly, blood vessels are segmented manually; however, this process is time-consuming, and the results can vary across users. Due to these limitations, automatic segmentation methods are preferable, particularly in tortuous cerebral vessels. 4D flow MRI provides both phase and signal magnitude data which can be used for image segmentation. Many automated segmentation methods make use of the 4D flow signal magnitude image, e.g., the pseudo-complex difference (PCD) intensity method considers the measured velocity's speed and magnitude to segment vessels. Despite its frequent use in segmentation, others have demonstrated that the signal magnitude image varies greatly throughout the field-of-view (FOV) depending on the flip angle of the MR scan. This variability of the magnitude image suggests that the performance of magnitude-based segmentation methods could also be inconsistent across the FOV. 4D flow MRI measurements can also be segmented according to the velocity time series. A number of these methods consider the pulsatile nature of biological fluid flows.

Unfortunately, this implies that many such methods are inapplicable to velocity profiles resembling steady flow (e.g., venous flow). These approaches also frequently require prior knowledge of the waveform to serve as a reference. In recent years, several deep learning approaches have been proposed to automatically segment PC-MRI data. These deep learning methods may not be generalizable to other parts of the vascular system or different MR sequences. Simpler statistical methods may prove to generalize to a broader range of applications.

### SUMMARY

The invention provides systems and methods to automatically segment flow data, such as but not limited to 4D flow magnetic resonance imaging (MRI) by identifying net flow effects according to the standardized difference of means (SDM) velocity. The SDM velocity quantifies the ratio between the net flow and observed flow pulsatility in each voxel. Vessel segmentation is performed using an F-test by identifying voxels with significantly higher SDM velocity values than tissue voxels. P-values for each voxel are reported to estimate the segmentation accuracy. We compare the SDM segmentation algorithm against pseudo-complex difference (PCD) intensity segmentation in in vitro scaled flow phantoms of a cerebral aneurysm and the in vivo cerebral vasculature of ten patients. The in vitro flow phantom geometry is known. In vivo, the ground truth geometries are derived from high-resolution time-of-flight (TOF) magnetic resonance angiography (MRA). The SDM segmentation algorithm is resilient to error sources within flow data, such as 4D flow MRI error sources. Qualitative results indicate that the SDM method performs well in regions with low velocity to noise ratios (VNR). The systems and methods herein demonstrate an approximate 48% increase in sensitivity in vitro and 70% in vivo compared to the PCD approach and is robust to a limited number of time frames per cardiac cycle. The vessel surface derived from the SDM method was 46% closer to the in vitro surfaces and 72% closer to the in vivo TOF MRA surfaces than the PCD approach. The SDM algorithm is a repeatable method to segment vessels, enabling reliable computation of hemodynamic metrics associated with cardiovascular disease.

In certain aspects, the invention provides methods for performing vessel segmentation from flow data representative of flow within a vessel, such as 4D flow Magnetic Resonance Imaging (MRI) flow data, that involve receiving flow data (such as 4D MRI flow data); identifying net flow effects in the flow data (such as 4D MRI flow data) according to a standardized difference of means (SDM) velocity that involves quantifying a ratio between net flow and observed flow pulsatility in each voxel of the received flow data (such as 4D MRI flow data); and identifying voxels with higher SDM velocity values than stationary tissue voxels, thereby performing vessel segmentation from flow data (such as 4D MRI flow data).

In other aspects, the invention provides systems for performing vessel segmentation from flow data representative of flow within a vessel, such as 4D flow Magnetic Resonance Imaging (MRI) flow data. The systems of the invention include a processor configured to: receive flow data (such as 4D MRI flow data); identify net flow effects in the flow data (such as 4D MRI flow data) according to a standardized difference of means (SDM) velocity that involves quantifying a ratio between net flow and observed flow pulsatility in each voxel of the received flow data (such as 4D MRI flow data); and identify voxels with higher SDM velocity values than stationary tissue voxels, thereby performing vessel segmentation from flow data (such as 4D MRI flow data).

In certain embodiments of the methods and systems of the invention, the systems and methods further involve generating a P-value for each voxel to estimate segmentation accuracy. In certain embodiments of the methods and systems of the invention, the SDM velocity, Ũᵢ, is defined as a difference between a time-averaged measured velocity at each voxel and a mean tissue velocity (\hat{μ}ᵢ) relative to a standard error,

$${stddev}_{t} [ {U}_{i , M} ( \vec{X} , t ) ] / \sqrt{{N}_{t}} : {\tilde{U}}_{i} ( \vec{X} ) = \frac{{mean}_{t} [ {U}_{i , M} ( \vec{X} , t ) ] - {\hat{μ}}_{i}}{{stddev}_{t} [ {U}_{i , M} ( \vec{X} , t ) ] / \sqrt{{N}_{t}}} .$$

In certain embodiments of the methods and systems of the invention, wherein SDM segmentations are generated by identifying voxels with significant values of Ũᵢ compared to tissue.

In certain embodiments of the methods and systems of the invention, the systems and methods further involve initially providing an approximation of tissue voxel locations. In certain embodiments of the methods and systems of the invention, the systems and methods further involve iteratively refining the vessel segmentation based on significant values of the SDM velocity. In certain embodiments of the methods and systems of the invention, the systems and methods further involve removing erroneous voxels from converged segmentation. In certain embodiments of the methods and systems of the invention, the systems and methods further involve incorporating near-wall voxels into the SDM segmentation.

In certain embodiments of the methods and systems of the invention, the systems and methods further involve using the vessel segmentation results to assess biomarkers of disease. In certain embodiments of the methods and systems of the invention, the disease is cardiovascular disease.

### BRIEF DESCRIPTION OF THE DRAWINGS

FIG. 1 shows the Standardized Difference of Means (SDM) segmentation algorithm detects voxels with significantly different values of SDM velocity than that of only tissue in the “initialization” and “iterative” steps. Upon convergence of the iterative step, the segmentation is post-processed in the “vessel isolation” and “dilation” steps. P-values at all voxels indicate the significance of the statistical test. User input parameters are indicated using red text. Herein, we interchangeably use the terms “segmentation” and “mask”.

FIG. 2 shows maps of SDM velocity magnitude (top panels) and p-values (bottom panels) associated with each voxel in a cross-sectional slice through the 1-to-1 (left panels) and 2-to-1 (right panels) phantom geometries. The outer boundaries of the benchmark segmentation are shown in green. P-values are expressed relative to the critical p-value in decibels.

FIG. 3 panels A-D show segmentation boundaries for the 1-to-1 (panels A and C) and 2-to-1 (panels B and D) in vitro phantom comparing PCD (panels A and B) and SDM (panels C and D) methods to the benchmark phantom geometries. PCD and SDM segmentations are shown as red surfaces, and the benchmark geometry is shown using blue wireframes.

FIG. 4 shows split violin plots comparing the relative minimum distance between predicted and benchmark flow phantom segmentations. The flow phantom scale is shown according to the position on the x-axis, and the method to predict the segmentation is shown on opposite sides of the split violin plot.

FIG. 5 panels A-C show cross-section through Patient H's Circle of Willis (panel A) passing through both ICAs, MCAs, and the 2.1 cm diameter aneurysm located on the right ICA. The SDM velocity magnitude and relative p-values along this cross-section are shown in panels B and C, respectively. The outer boundaries of the TOF segmentation are shown in green.

FIG. 6 panels A-B show segmentation boundaries for Patient H with a 2.1 cm diameter aneurysm on the right ICA. Panel A) compares the PCD method and TOF, and panel B) compares the SDM method and TOF. PCD and SDM segmentations are shown using red surfaces, and TOF is shown using blue wireframes.

FIG. 7 shows split violin plots comparing the relative minimum distance between predicted and benchmark segmentations. The patient identifier is shown according to the position on the x-axis and the method to predict the segmentation is shown on opposite sides of the split violin plot.

FIG. 8 shows an example of a vessel segmentation (shown in red) generated from time-of-flight (TOF) angiography. The TOF data was segmented semi-automatically using ITK-SNAP.

FIG. 9 shows an example of an existing automatic vessel segmentation (red) compared to the actual vessel locations (grey). Existing automatic segmentation methods erroneously include regions of high noise and exclude vessels partially or entirely.

FIG. 10 shows a map of SDM velocity magnitude associated with each voxel in a cross-sectional slice through the 2-to-1 in vitro flow phantom. The outer boundaries of the actual segmentation are shown in green. The velocity was measured using 4D flow MRI.

### DETAILED DESCRIPTION

This invention provides systems and methods that utilize an automatic segmentation algorithm that operates on the 4D flow measured velocity field. In certain aspects, the invention provides the “standardized difference of means (SDM) velocity” metric, which identifies voxels exhibiting velocity characteristics that are significantly different than those found in stationary tissue. The systems and methods herein also provide a p-value for each voxel to estimate the segmentation accuracy. The performance of the SDM segmentation was compared against the PCD algorithm in in vitro scaled aneurysm flow phantoms and in vivo measurements of ten patients' cerebral vasculatures. The benchmark in vitro flow phantom geometries and the in vivo high-resolution time-of-flight (TOF) magnetic resonance angiography (MRA) derived geometries serve as the ground truth for assessing SDM and PCD segmentation performance.

In the following sections we describe the SDM segmentation method and studies performed to validate the proposed algorithm. Herein we describe the SDM velocity and its application to segmenting vessels. Next, we outline the SDM segmentation algorithm, which features the SDM velocity. Individual steps in the algorithm are described. The in vitro and in vivo studies are outlined, respectively. Performance metrics applied in this work to compare the SDM and PCD segmentations are presented.

Standardized Difference of Means (SDM) Velocity We denote the 4D flow measured velocity field as U_{i,M}, which is resolved in 3D space, \vec{X}∈ [symbol] , and time, t. The vector components along each spatial dimension are represented using the i subscript, such that i=x, y, z.

The SDM velocity, Ũᵢ, is defined as the difference between the time-averaged velocity at each voxel and the mean tissue velocity (\hat{μ}ᵢ) relative to the standard error,

$${stddev}_{t} ( {U}_{i , M} ) / \sqrt{{N}_{t}} : {\tilde{U}}_{i} ( \vec{X} ) = \frac{{mean}_{t} ( {U}_{i , M} ) - {\hat{μ}}_{i}}{{stddev}_{t} ( {U}_{i , M} ) / \sqrt{{N}_{t}}} \qquad ( 1 )$$

where Nₜ is the total number of acquired time frames.

We assume the measured velocity is primarily the effect of blood flow (U_{i,F}) and velocity noise (ρᵢ). We also assume that 4D flow MRI velocity noise is additive, U_{i,M}=U_{i,F}+ρᵢ. The motion of the vessel wall is assumed to be less than the size of the voxel. Limited wall motion is widely assumed when studying flow in the brain. As a result, we expect U_{i,M} to only be due to noise effects in tissue, U_{i,M} (Tissue)=ρᵢ(Tissue).

In (1), Ũᵢ is t-distributed only if the measured velocity is uncorrelated. Despite spatiotemporal covariance due to the parallel imaging techniques, in tissue, Ũᵢ approaches a zero-mean normal distribution with variance

$${σ}_{\tilde{U}}^{2}$$

as Nₜ increases according to the central limit theorem:

$${\tilde{U}}_{i} ∼ 𝒩 ( 0 , {σ}_{\tilde{U}}^{2} ) ,$$

when \vec{X}∈ Tissue (2)

Any non-zero mean present in the noise (e.g., from Eddy currents) is removed by subtracting \hat{μ}ᵢ in (1) [13], [47]. Equation (2) will serve as the null hypothesis in the SDM segmentation algorithm. Significant values of Ũᵢ are assumed to be the result of blood flow.

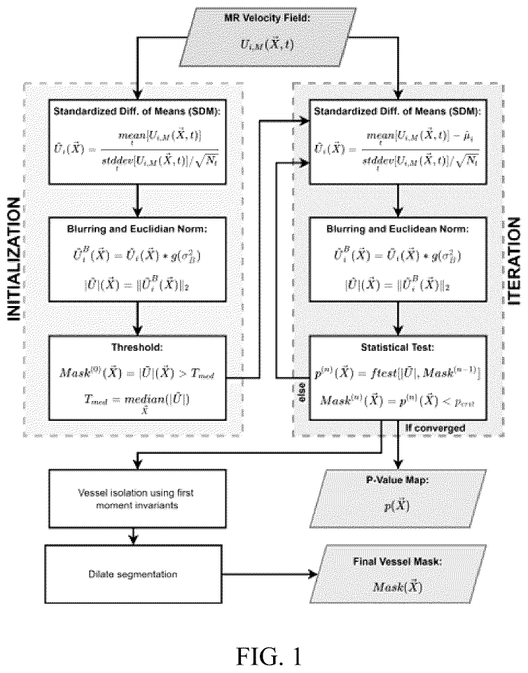

FIG. 1 presents the SDM segmentation algorithm, which generates a mask of 4D flow MRI measurements. P-values are provided to express the significance of flow effects at all voxels in the image volume. Herein, we interchangeably use the terms “segmentation” and “mask.”

The “initialization” step provides a crude approximation of tissue voxel locations (Section II-B-1). The “iteration” step estimates

$${σ}_{\tilde{U}}^{2}$$

for each 4D flow MRI dataset according to the measured velocity in tissue voxels (Section II-B-2). We then infer if the observed value of Ũᵢ at each voxel is significantly different than that expected of only noise effects found in the tissue. Voxels with a significant difference are presumed to exhibit flow and are included in the vessel segmentation. The “vessel isolation” and “dilation” steps (discussed below) constitute post-processing steps.

### Initialization

The SDM algorithm is initialized using ûᵢ=0 when calculating Ũᵢ. Euclidean norm and blurring operations are applied in the SDM algorithm to improve the detection of voxels with net flow and reduce the SDM velocity variance in tissue voxels. The blurred SDM velocity is calculated by convolving all components of Ũᵢ with a Gaussian blurring kernel according to a user-defined variance

$${σ}_{B}^{2} ( g ( 0 , {σ}_{B}^{2} ) ) .$$

we generally recommend setting

$${σ}_{B}^{2} = 1 .$$

For images with low saturation ratio (SR≤0.1), we recommend

$${σ}_{B}^{2} < 1$$

as there is already sufficient contrast between flow and tissue voxels. The magnitude of the blurred SDM velocity, |Ũᵢ| is then evaluated. The initialized mask, Mask⁽⁰⁾, constitutes all voxels with |Ũᵢ| values greater than the median value of |Ũᵢ| across the entire FOV,

$${Mask}^{( 0 )} = \vert {\tilde{U}}_{i} \vert > {median}_{{X}^{→︀}} ( \vert {\tilde{U}}_{i} \vert ) .$$

### Iteration

The iteration step refines the mask provided by the initialization phase until there are no changes in the segmentation, as shown in FIG. 1. Many steps in the iteration step match those described herein, including the blurring and Euclidean norm steps. However, the iteration phase evaluates Ũᵢ after calculating ûᵢ, the arithmetic mean across all time frames and all voxels outside of the past mask iteration, n−1, such that

$${\hat{μ}}_{i} = {mean}_{{\vec{X} ∉ {Mask}^{( n - 1 )}}_{t}} ( {U}_{i , M} ) .$$

We evaluate |Ũᵢ| for all voxels in the FOV as in the initialization phase; however, we generate the mask for each iteration using an F-test. To perform this statistical test, we first evaluate the F-test statistic, F*, for each voxel in the FOV:

$$F * ( \vec{X} ) = \frac{SSR ( \vec{X} ) / {df}_{R}}{SSE / {df}_{E}} \approx \frac{{\vert {\tilde{U}}_{i} \vert}^{2} ( \vec{X} )}{{σ}_{\tilde{U}}^{2}} \qquad ( 3 )$$

where SSR and SSE are the regression and error sum of squares, respectively, with degrees of freedom df_{R} and df_{E}. We evaluate SSR for each voxel as SSR(\vec{X})=[|Ũᵢ|(\vec{X})²] for df_{R}=1. We calculate SSE using all voxels outside of the mask from past iteration, SSE=Σ_{\vec{X}∉Mask}_{⁽ⁿ⁻¹⁾}[|Ũᵢ|(\vec{X})]², with df_{E}=N_{Tiss}−1 where N_{Tiss} is the number of voxels outside of the mask. We approximate df_{E} as N_{Tiss}−1 by assuming the local noise correlations are negligible in comparison to the total noise variance across the FOV. The subtraction of one from N_{Tiss} results from the calculation of sample means when evaluating the sum of squares values. F* is approximately equal to

$${\vert {\tilde{U}}_{i} \vert}^{2} / {σ}_{\tilde{U}}^{2} as SSE / {df}_{E}$$

is a measure of the sample variance.

We evaluate the p-value at each voxel, p(\vec{X}), according to F*, df_{R}, and df_{E}. This p-value quantifies the probability that noise effects describe the observed variance of |Ũᵢ|. The mask for the current iteration is generated as p-values that are lower than the user-defined critical p-value, p_{crit}, Mask⁽ⁿ⁾(\vec{X})=p⁽ⁿ⁾(\vec{X})<p_{crit}. Herein, p_{crit} is 0.01 for all cases.

We continue the iteration process until the number of voxels in the mask is unchanged. Upon convergence, p(\vec{X}) serves a p-value map for the final SDM segmentation.

### Vessel Isolation

The “vessel isolation” step in the SDM segmentation algorithm aims to remove erroneous voxels from the converged mask. Despite satisfying the null hypothesis, a relatively small number of tissue voxels reside far in the tails of the distribution described by (2), exhibiting significant SDM velocity.

To isolate voxels that segment vessels, we first label all separate regions of the converged SDM mask. We break the converged mask up into individual regions, [symbol] , with q being the region index. We define each region as a set of voxels connected according to 6-connectivity using the scipy.ndimage.label method in Python.

We expect vessel regions to have a higher number of voxels and to extend across the FOV, unlike erroneous voxel regions. We quantify this property of each region using the first moment invariant,

$${J}_{1}^{( q )} .$$

We automatically select the regions to retain in the SDM segmentation according to a mean threshold of all values of

$${J}_{1}^{( q )} , Mask = {J}_{1}^{( q )} > {mean}_{q} ( {J}_{1}^{( q )} ) .$$

### Dilation

The dilation step aims to incorporate partial volume (PV) voxels in the SDM segmentation. PV voxels tend to exhibit lower values of Ũᵢ than core flow (CF) voxels because PV voxels partially contain the vessel wall and surrounding tissue. Tissue has a velocity of zero, and blood flow near the vessel wall is generally lower than in CF voxels due to the no-slip condition. This claim regarding Ũᵢ in PV voxels is demonstrated in Section III. PV voxels are incorporated by dilating the segmentation by a single voxel using Python's skimage.morphology.binary_dilation method.

In Vitro 4D flow in Scaled ICA Aneurysms

The SDM segmentation algorithm is applied to in vitro 4D flow MRI measurements in 3D-printed flow phantoms replicating an internal carotid artery (ICA) aneurysm. According to IRB-approved protocol, in vivo 4D flow and TOF MRI data of the cerebral vasculature were acquired at Northwestern University. Two flow phantoms were fabricated, one matching in vivo dimensions (unscaled) and one scaled in all directions by a factor of two (scaled). The flow phantom geometries were generated from TOF data, segmented with ITK-SNAP and post-processed using Geomagic Design software (3D Systems, Rock Hill, SC) to model the aneurysm's luminal surface. The 1-to-1 and scaled-up 2-to-1 in vitro phantoms were fabricated with a high-resolution ProJet MJP 2500 Plus 3D printer (3D Systems, Rock Hill, SC).

A flow loop was created to conduct in vitro 4D flow measurements of the intra-aneurysmal flow at a steady flow rate. The working fluid was a water-glycerol mixture (60:40 by volume). The Reynolds number in the two in vitro geometries was matched to ensure flow similarity. In vitro velocity measurements in both phantoms were acquired at Purdue University's MRI Facility on a Siemens 3T PRISMA scanner using dual-venc 4D flow MRI, which applies k-t GRAPPA acceleration [49]. Parameters used for each phantom scale are shown in Table I. Additional details of this experiment are provided in our previous work.

TABLE I
IMAGING PARAMETERS FOR IN VITRO
SCALED ANEURYSM STUDIES

| | VENCS | VOXEL SIZE | TIME | TE, TR |
|---|---|---|---|---|
| SCALE | (cm/s) | (mm³) | FRAMES | (ms) |
| 1-to-1 | 30, 60 | 0.80 × 0.80 × 0.80 | 18 | 4.08, 6.78 |
| 2-to-1 | 15, 30 | 0.85 × 0.85 × 0.80 | 18 | 4.80, 7.50 |

We apply the SDM segmentation algorithm to the 1-to-1, and 2-to-1 scaled geometries using the 4D flow MRI velocity measurements. In both phantoms, we set the blurring coefficient,

$${σ}_{B}^{2} = 0 ,$$

and the critical p-value, p_{crit}=0.01. We compare our SDM segmentation algorithm to segmentations generated using a pseudo-complex difference (PCD) method. PCD segmentations serve as the reference for our SDM algorithm as it does not require model training and is fully automatic when coupled with dynamic thresholding. The PCD algorithm can also be applied to cases of steady flow.

To assess the PCD segmentation, we first evaluate the PCD intensity as defined by Schnell et al. [49]:

$${I}_{PCD} = \{ \begin{matrix}M sin ( \frac{π \vert {U}_{i , M} \vert}{venc} ) \vert {U}_{i , M} \vert , & when \vert {U}_{i , M} \vert < \frac{2 venc}{3} \\ M , & otherwise\end{matrix} \} \qquad ( 4 )$$

where M is the signal magnitude of the 4D flow MRI data, and |U_{i,M}| is the speed of the 4D flow MRI measured velocity. I_{PCD} is resolved in space and time. We evaluate the 3D I_{PCD} resolved only in space by calculating the mean squares with respect to time as Berhane et al. [32]. In typical practice, segmentations are generated from the PCD intensity according to a user-defined threshold or manual segmentation [49]. To eliminate user influence in PCD segmentation generation and promote direct comparisons between the PCD and SDM methods, we automatically threshold the PCD intensity using a triangle threshold. The automatic triangle threshold operation is performed in Python's scikit-image library using skimage.filters.threshold triangle.

Since the flow phantoms were 3D-printed, the true vessel geometries are known. We determine the accuracy of the SDM and PCD segmentations by registering the true flow phantom geometry to the segmentations using the Coherent Point Drift (CPD) algorithm [50], [51]. The true segmentations are assessed by downsampling the high-resolution 3D-printed geometries to the resolution of the 4D flow MRI measurements.

We qualitatively assess the performance of the segmentation methods by comparing the surfaces of the SDM and PCD to that of the benchmark segmentation provided by the phantom geometry. Maps of the SDM velocity and p-values will be generated to demonstrate the method's sensitivity to regions of flow. Quantitative comparisons will be made using performance metrics described herein.

In Vivo 4D flow in the Circle of Willis

The performance of the SDM segmentation algorithm will be assessed in the Circles of Willis of ten patients, with TOF-based segmentation serving as a benchmark. According to IRB-approved protocol, in vivo 4D flow MRI and TOF data of the cerebral vasculature were acquired at Northwestern University. Relevant parameters of the 4D flow MRI data for all ten patients are shown in Table II. All patient scans had flip angles of 15 degrees. The TOF voxel sizes for Patients A, E, I, and J were 0.60×0.43×0.43 mm³, 0.60×0.27×0.27 mm³, 0.50×0.52×0.52 mm³, and 0.55×0.26×0.26 mm³, respectively. All other patients had TOF voxel sizes of 0.50×0.26× 0.26 mm³.

The 4D flow images were segmented using the SDM algorithm for

$${σ}_{B}^{2} = 1 and {p}_{crit} = 0.01$$

in all ten patients. The PCD segmentations were generated using the procedure described herein.

TABLE II
4D FLOW MRI PARAMETERS FOR IN VIVO STUDIES

| | VENCS | VOXEL SIZE | TIME | TE, TR |
|---|---|---|---|---|
| PAT. | (cm/s) | (mm³) | FRAMES | (ms) |
| A | 50, 100 | 1.15 × 1.15 × 1.20 | 16 | 3.40, 6.10 |
| B | 50, 100 | 1.04 × 1.04 × 1.00 | 11 | 3.48, 6.30 |
| C | 50, 100 | 1.15 × 1.15 × 1.20 | 8 | 3.39, 6.10 |
| D | 50, 100 | 1.04 × 1.04 × 1.20 | 6 | 3.39, 6.10 |
| E | 60, 120 | 0.98 × 0.98 × 1.00 | 17 | 3.40, 6.19 |
| F | 50, 100 | 1.15 × 1.15 × 1.20 | 16 | 3.39, 6.10 |
| G | 50, 100 | 1.15 × 1.15 × 1.20 | 16 | 3.39, 6.10 |
| H | 50, 100 | 0.98 × 0.98 × 1.00 | 16 | 3.52, 6.30 |
| I | 60, 120 | 0.98 × 0.98 × 1.00 | 19 | 3.40, 6.20 |
| J | 60, 120 | 1.04 × 1.04 × 1.00 | 15 | 3.36, 6.10 |

The leftmost column indicates the unique identifier for each patient (pat.)

The TOF images were segmented as in Section II-C to serve as a reference for assessing SDM and PCD segmentation accuracy [48]. We determine the accuracy of the SDM and PCD segmentations by registering the TOF data to the segmentations using the CPD algorithm as in Section II-C. The registered TOF segmentations are then downsampled to the resolution of the 4D flow MRI measurements. We limited the downsampled TOF to the Circle of Willis by removing other vessels passing through the edge of the FOV.

Patient H's Circle of Willis exhibits a 2.1 cm diameter aneurysm on the right internal carotid artery (ICA). The slow flow in large aneurysms is typically difficult for flow-based segmentation methods to segment and will demonstrate the robustness of the SDM segmentation algorithm. We qualitatively assess the performance of the segmentation methods by comparing the surfaces of the SDM and PCD to the downsampled TOF segmentations in Patient H. Maps of the SDM velocity and p-values found for Patient H were generated to demonstrate the method's detection of flow regions. Quantitative comparisons across all patients will be made using performance metrics described herein.

### Performance Metrics

We quantitatively assess the performance of the predicted segmentations generated by the SDM or PCD algorithms in reference to the benchmark segmentations. These performance metrics explore the distance between the predicted and benchmark segmentation surfaces and the overlap of the predicted and benchmark segmentation volumes. The benchmark segmentations are provided by the true in vitro geometry or the in vivo TOF segmentations.

We evaluate the segmentation accuracy by calculating the minimum distances from each voxel on the benchmark surface to the predicted vessel surface. We express the locations of benchmark and predicted surface segmentation voxels as \vec{Y}_{B} and \vec{Y}_{P}, respectively. The minimum distance between \vec{Y}_{B} and \vec{Y}_{P}, for all \vec{Y}_{B} is written as:

$${d}_{min} ( {\vec{Y}}_{B} ) = min ( {‖ {\vec{Y}}_{B} - {\vec{Y}}_{P} ‖}_{2} ) \qquad ( 5 )$$

As a result, a distribution of dₘᵢₙ is generated for each phantom and patient.

We evaluate the predicted positives (PP) and predicted negatives (PN) from the SDM or PCD segmentation volumes to evaluate overlap metrics. We also evaluate benchmark positives (BP), and benchmark negatives (BN) assessed using the benchmark in vitro geometry or in vivo TOF. Using these predicted and benchmark segmentations, we assess the number of true positives (TP), false positives (FP), false negatives (FN), and true negatives (TN). Table III shows that these quantities are evaluated as the intersection of the appropriate predicted and benchmark segmentations:

TABLE III
SEGMENTATION PERFORMANCE METRICS

| METRIC | DEFINITION |
|---|---|
| True Positives | TP = PP ∩ BP |
| False Negatives | FN = PN ∩ BP |
| False Positives | FP = PP ∩ BN |
| True Negatives | TN = PN ∩ BN |
| Sensitivity | Sen = TP/BP |
| Specificity | Spec = TN/BN |
| Precision | Prec = TP/PP |
| Negative Predictive Value | NPV = TN/PN |
| Balanced Accuracy | Bal Acc = (Sen + Spec)/2 |

Performance metrics assessed from the predicted positives (PP), benchmark positives (BP), predicted negatives (PN), and benchmark negatives (BN).

We look to calculate relative quantities so the performance of the SDM and PCD algorithms can be directly compared across in vitro geometry scales and in vivo patient data. To achieve this, we evaluate the sensitivity (Sen.), specificity (Spec.), precision (Prec.), negative predictive value (NPV), and balanced accuracy (Bal. Acc.) as defined in Table III.

The balanced accuracy is evaluated in cases where the performance in assessing negatives and positives should be equally represented [52]. Our datasets generally contain far more benchmark negatives than benchmark positives because blood vessels make up a small fraction of the overall image volume.

In Vitro 4D flow in Scaled ICA Aneurysm

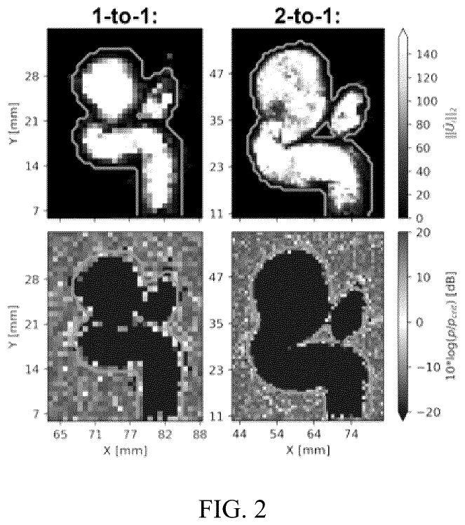

The SDM algorithm segments voxels which exhibit significantly high values of SDM velocity. FIG. 2 plots cross-sections of the SDM velocity magnitude and p-values in the in vitro flow phantoms. We express p-values relative to the critical p-value in decibels to best express the range of p-values. These cross-sectional planes cut through the ICA inlet, ICA outlet, and aneurysm sac. The green outline indicates the surface of the down-sampled benchmark segmentation of the 3D-printed flow phantoms.

In both the 1-to-1 and 2-to-1 flow phantoms, the SDM magnitudes are much higher inside the phantom geometries than outside. This high contrast of the SDM velocity produces very low p-values inside the segmentation. This is demonstrated in FIG. 2 by the relative p-values being less than-20 dB inside the segmentation. We observe that SDM magnitude values decrease as the voxels in the flow approach the wall of the flow phantom. This leads to increased p-values near the wall as well. Voxels near the wall are partially composed of tissue and experience slower flow due to the no-slip condition.

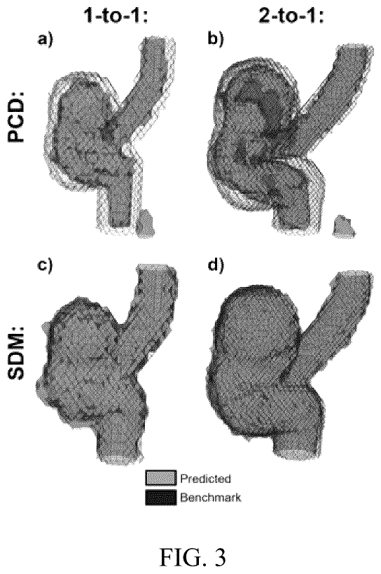

FIG. 3 panels A-D show the PCD (panels A and B) and SDM segmentations (panels C and D) in reference to the benchmark flow phantom geometries in the 1-to-1 (panels A and C) and 2-to-1 (panels B and D) geometries. Red surfaces indicate the PCD or SDM segmentations, and the blue wireframes show the true geometry.

In both the 1-to-1 and 2-to-1 datasets, the PCD segmentation omits many near-wall voxels which are captured in the SDM segmentations. Furthermore, the PCD segmentation of the 2-to-1 phantom (panel B) misses a region in the center of the aneurysm sac. In contrast, these voxels are included in the SDM segmentation (panel D). Panels a and b show that the PCD algorithm erroneously includes a segmented region to the right of the ICA inlet, resulting from a phase wrapping artifact. The SDM algorithm correctly omits this artifact.

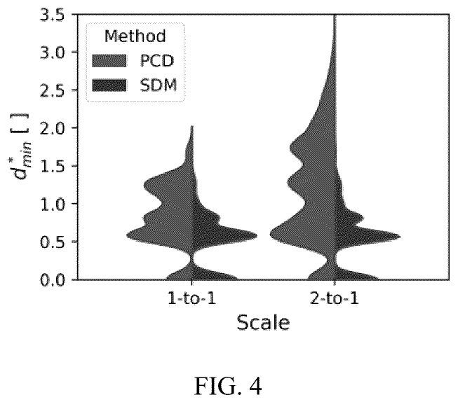

We quantitatively assess the discrepancy between the surfaces of the predicted and benchmark segmentation using the minimum distance performance metric, dₘᵢₙ, described herein. To promote comparison of minimum distances across segmentations, we non-dimensionalize dₘᵢₙ using the L2-norm of the acquisition's voxel size,

$${d}_{min}^{*} = {d}_{min} / {‖ {δ}_{i} ‖}_{2} ,$$

where δᵢ is the 3D voxel size. FIG. 4 plots the distributions of

$${d}_{min}^{*}$$

according to un How phantom scale and segmentation method.

The violin plots exhibit many peaks as

$${d}_{min}^{*}$$

is a discrete variablee. The SDM segmentation surfaces are equidistant or closer to the benchmark segmentation in 77.8% of voxels compared to the PCD surfaces. Furthermore, when comparing different phantom scales, E the SDM method produces similar distributions of

$${d}_{min}^{*} .$$

Contrastingly, the PCD method exhibits a greater spread of

$${d}_{min}^{*}$$

in the 2-to-1 phantom compared to 1-to-1. These findings are quantitatively expressed in Table IV, which presents the median and RMS

$${d}_{min}^{*}$$

values according to the segmentation method and in vitro geometry scale.

The SDM method exhibits lower median and RMS

$${d}_{min}^{*}$$

values than the PCD method for both in vitro scales. The SDM method shows more conserved median and RMS values between the scaled geometries with differences of 0.03 and 0.06, respectively, as compared to the PCD method, which exhibited differences of 0.36 and 0.46. Overall, the SDM segmentations exhibit medians and RMS values, respectively, 42.0% and 49.6% lower than the PCD approach.

TABLE IV
IN VITRO MINIMUM RELATIVE DISTANCE

| | |
|---|---|
| PCD | SDM |

| SCALE | MEDIAN | RMS | MEDIAN | RMS |
|---|---|---|---|---|
| 1-to-1 | 0.82 | 0.92 | 0.58 | 0.60 |
| 2-to-1 | 1.18 | 1.38 | 0.55 | 0.66 |
| Total | 1.00 | 1.29 | 0.58 | 0.65 |

We explore the SDM method's performance in assessing the full segmentation volume using overlap metrics. Table V presents these metrics according to the in vitro phantom scale and segmentation method.

The SDM method demonstrates a 47.83% and 48.08% increase in sensitivity compared to the PCD method for the 1-to-1 and 2-to-1 phantom scales, respectively. The SDM method indicates a 23.24% and 23.34% increase in balanced accuracy compared to the PCD method for the 1-to-1 and 2-to-1 phantom scales, respectively. However, Table V shows that the SDM method is less precise than the PCD segmentation algorithm in this in vitro study. Values of NPV and specificity are similar across segmentation methods and aneurysm phantom scales.

TABLE V
IN VITRO OVERLAP METRICS

| | |
|---|---|
| PCD | SDM |

| METRIC | 1-TO-1 | 2-TO-1 | 1-TO-1 | 2-TO-1 |
|---|---|---|---|---|
| Sen. | 49.77% | 49.24% | 97.67% | 97.32% |
| Spec. | 99.95% | 99.89% | 98.53% | 98.29% |
| Prec. | 97.23% | 96.85% | 69.05% | 79.67% |
| NPV | 98.35% | 96.60% | 99.92% | 99.81% |
| Bal. Acc | 74.86% | 74.56% | 98.07% | 97.80% |

Performance values are bolded when they are decidedly higher
than alternate segmentation method.

### In Vivo 4D Flow in ICA Aneurysm

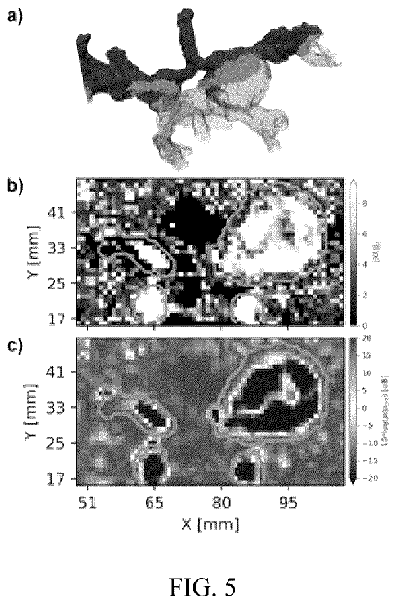

The performance of the SDM method is assessed in vivo in the cerebral vasculature of ten patients. FIG. 5 panels A-C show the SDM velocity magnitude and p-values in a cross-section of Patient H's Circle of Willis. FIG. 5 panel A depicts the location of this cross-section as a green plane that passes through both ICAs, middle cerebral arteries (MCAs), and the 2.1 cm diameter aneurysm located on the right ICA. The green outline indicates the surface of the down-sampled TOF segmentation in FIG. 5 panels B-C.

The SDM magnitude in FIG. 5 panel B appears higher within the TOF segmentation than in the surrounding tissue. This SDM contrast promotes low p-values inside the segmentation (FIG. 5 panel C). SDM magnitude values decrease as the voxels in the flow approach the wall of the flow phantom. This leads to increased p-values near the wall. The intra-aneurysmal voxels exhibit lower SDM values at x=85, y=33 with a trail of low values extending to x=95, y=37. This trail of low SDM values corresponds to higher relative p-values shown in FIG. 5 panel C.

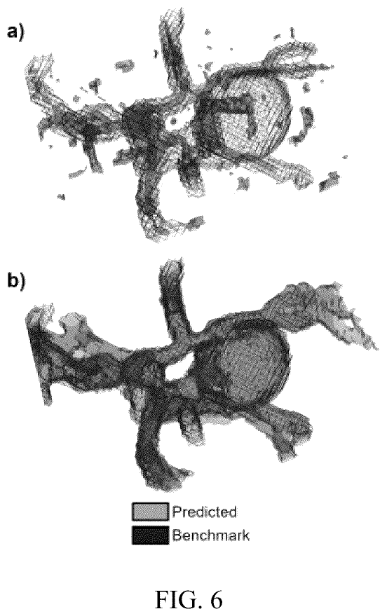

FIG. 6 panel A shows the PCD segmentation in reference to the down-sampled TOF segmentation. The SDM segmentation and TOF segmentation are shown in FIG. 6 panel B. The PCD segmentation omits many voxels near the wall and most of the voxels in the aneurysm sac are excluded from the segmentation. Contrastingly, most of these near-wall and intra-aneurysmal voxels are captured by the SDM algorithm. This suggests that the SDM method is more sensitive to slow blood flow regions than the PCD method. The PCD method also includes many tissue voxels with high noise, omitted by the SDM algorithm. The SDM segmentation includes vessels that are not part of the TOF segmentation. These vessels are likely veins that are suppressed during the TOF acquisition.

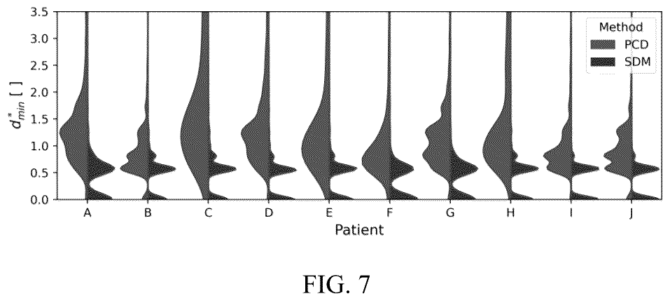

FIG. 7 quantitatively expresses the differences between the PCD and SDM segmentation surfaces compared to TOF for all ten patients. As in the above, surface voxel performance is assessed using the relative minimum distance between the benchmark and predicted segmentations,

$${d}_{min}^{*} .$$

TABLE VI
IN VIVO MINIMUM RELATIVE DISTANCE

| | |
|---|---|
| PCD | SDM |

| PATIENT | MEDIAN | RMS | MEDIAN | RMS |
|---|---|---|---|---|
| A | 1.27 | 1.70 | 0.57 | 0.76 |
| B | 0.81 | 1.00 | 0.56 | 0.58 |
| C | 1.70 | 3.29 | 0.57 | 0.63 |
| D | 1.23 | 1.70 | 0.55 | 0.53 |
| E | 1.15 | 2.75 | 0.57 | 0.56 |
| F | 0.82 | 1.53 | 0.57 | 0.67 |
| G | 1.00 | 1.29 | 0.57 | 0.70 |
| H | 1.17 | 2.19 | 0.57 | 0.54 |
| I | 0.82 | 1.10 | 0.57 | 0.47 |
| J | 0.81 | 1.00 | 0.56 | 0.45 |
| Total | 1.14 | 2.10 | 0.56 | 0.59 |

The SDM segmentation method demonstrates lower values of

$${d}_{min}^{*}$$

compared the PCD method across all ten patients. The SDM segmentation surfaces are equidistant or closer to the benchmark segmentation in 93.5% of voxels compared to the PCD surfaces. Moreover, extended upper tails observed for the PCD method are absent from the SDM segmentations. Median and RMS values for each distribution shown in FIG. 7 are presented in Table VI.

For each patient, the SDM method exhibits lower median and RMS values than the PCD method. Furthermore, the median and RMS values are more similar using the SDM method than the PCD method, which suggests fewer outliers. The range of median values for the SDM and PCD methods is 0.02 and 0.89, respectively. The range of RMS values is 0.31 and 2.29 for the SDM and PCD methods, respectively. Overall, the median and RMS values of

$${d}_{min}^{*}$$

are 50.9% and 71.9% lower in vivo for SDM segmentations than those from the PCD method.

TABLE VII
IN VIVO OVERLAP METRICS FOR ALL PATIENTS

| METRIC | PCD | SDM |
|---|---|---|
| Sen. | 16.49% | 86.63% |
| Spec. | 99.71% | 94.73% |
| Bal. Acc | 58.10% | 90.68% |

Performance values are bolded when they are decidedly higher than
alternate segmentation method.

Table VII presents the overlap metrics according to the segmentation method for all ten patients. The SDM method demonstrates a 70.14% increase in sensitivity and a 32.58% increase in balanced accuracy compared to the SDM method in vivo. The specificity of the two methods is similar. Calculations of precision and NPV are not valid using TOF as a reference as this imaging modality does not capture veins when presaturation pulses are applied.

### Discussion

In this work, we present the SDM velocity as a feature for segmenting 4D flow MRI measurements. We embed the SDM velocity in an iterative algorithm to identify voxels with significant flow effects, which are identified by comparing the level of SDM velocity to that expected from tissue effects alone. Upon convergence of the segmentation, erroneously segmented voxels are removed by considering the first moment invariant of each region of connected voxels. Voxels with PV effects are added to the SDM segmentation using binary dilation. P-values are reported to express the significance of flow effects at all voxels. The performance of the SDM segmentation is demonstrated in reference to the PCD algorithm in in vitro scaled aneurysm flow phantoms and in vivo 4D flow measurements of cerebral vasculatures in ten patients. The 3D-printed in vitro flow phantom geometries and high-resolution in vivo TOF-derived geometries served as the ground truth for assessing the performance of our SDM segmentations and those obtained with PCD.

The SDM velocity in (1) expresses the ratio of net flow to the pulsatility measured by 4D flow MRI. In tissue, this SDM velocity follows the zero-mean normal distribution reported in (2). In contrast, voxels with net flow do not follow this same distribution. FIGS. 2 and 5 panels A-C show that voxels inside the benchmark segmentation generally have much higher SDM velocity magnitude values than those outside the segmentation. Our in vitro steady flow experiment demonstrates SDM velocity magnitude values two orders of magnitude higher than those observed in vivo. The in vitro steady flow exhibits very low spread of the measured velocity in time. According to (1), this low pulsatility is predicted to produce higher SDM velocity values than in vivo arterial flow. Tables V and VII quantitatively show that the SDM algorithm is more sensitive to voxels with flow than the PCD method in vitro and in vivo.

FIGS. 2 and 5 panels A-C also demonstrate that regions of high SDM velocity have low p-values and vice versa. This relationship between the SDM velocity and p-values is created by (3) as F* serves as the test statistic for evaluating p-values. In vitro and in vivo, near-wall voxels exhibited less significant flow effects compared to regions in the core of the flow. These less significant flow effects are expected due to the influence of tissue in the PV voxels and the slower blood flow at the wall. Our algorithm addresses the low significance of flow effects in PV voxels by implementing the dilation step. The elevated p-values in the center of the aneurysm observed in FIG. 5 panel C and reduced SDM velocity in the same region in FIG. 5 panel B indicates this is an area with low net flow, suggesting that this is the center of a vortex in the aneurysm.

The definition of the SDM velocity in (1) is robust to 4D flow MRI errors and addresses 4D flow's intra-scan variability (i.e., throughout the FOV). We observe consistent performance of the SDM algorithm in the FOV despite highly variable levels of noise owing to different tissue signal properties and variable SNR. FIGS. 3 panels A-D and 6 panels A-B show that the SDM segmentations better assess near-wall voxels affected by PV effects than the PCD algorithm.

FIGS. 4 and 7 show that the SDM algorithm presents narrower distributions or

$${d}_{min}^{*}$$

compared to PCD segmentations in vitro and in vivo for all ten patients. This demonstrates that the SDM segmentation algorithm more closely represents the vessel's surface throughout the FOV. Furthermore, FIGS. 3 panels C-D and 6 panel B show that the SDM segmentation correctly segments voxels inside the aneurysm, which were occasionally omitted by the PCD algorithm. These findings suggest that the SDM segmentation algorithm is robust to low VNR as observed in intra-aneurysmal flow. The robust nature of the SDM algorithm would enable more accurate computation of hemodynamic metrics, especially those relying on near-wall velocities, e.g., WSS, implicated in several cardiovascular pathologies.

The SDM algorithm uses the F-test statistic in (3) to address inter-scan variability caused by differences between patients and 4D flow scan settings. This standardized performance is achieved by dividing the SDM velocity by the observed tissue variance in (3), promoting direct comparison between different 4D flow scans. This uniform performance of the SDM algorithm is demonstrated in FIG. 7 by the consistent form of the SDM distributions across all ten patients as opposed to PCD. This is also shown quantitatively in Table VI according to the smaller range of median and RMS values across patients for the SDM method as compared to the PCD method. Despite Patient D only having 6 time frames (Table II), FIG. 7 and Table VI show that the SDM segmentation method performed similarly relative to other patients who had as many as 19 time frames (Patient I). The in vitro study demonstrates that the SDM segmentation is robust to limited spatial resolution. Table IV shows this quantitatively by the closer agreement in SDM median and RMS values across flow phantom scales relative to the PCD values.

The SDM algorithm segments all flow regions in the FOV, thus requires further post-processing steps for selecting arterial or veinous vasculature. Additionally, the SDM method assumes that any wall motion occurs within the scale of an image voxel. While this is a reasonable assumption in the brain, tissue motion is larger in other vascular regions [56], which may affect the performance of the algorithm in regions with more extensive wall motion. Furthermore, the SDM method inherently assumes voxels in blood vessels exhibit net flow across the cardiac cycle,

$${E}_{t} ( {U}_{i , M} ) \neq 0.$$

Areas in the vessel with low net flow (e.g., CSF flow) may be excluded from the SDM segmentation.

Our results suggest that the algorithm is limited by reduced precision despite yielding increased sensitivity. This behavior is not limited to our algorithm and is generally referred to as the “Precision-Recall Tradeoff” in classification methods. The SDM segmentation algorithm's design prefers low false negatives as opposed to low false positives.

This work presents an approach for segmenting vessels in 4D flow MRI measurements based on the SDM velocity. The SDM velocity quantifies the relation of net flow through the voxel to the flow pulsatility. The accuracy of the SDM segmentation algorithm is reported according to p-values at all voxels. Our segmentation algorithm is validated on 4D flow MRI measurements in vitro using flow phantom geometries and in vivo, using high-resolution TOF MR angiography in ten patients. The SDM algorithm serves as a repeatable method to segment vessels across a range of scan settings and imaging conditions. This consistent performance of the SDM method would enable more accurate computation of hemodynamic metrics, especially those relying on near-wall velocities such as wall shear stress, which have been implicated in various cardiovascular pathologies.

### INCORPORATION BY REFERENCE

References and citations to other documents, such as patents, patent applications, patent publications, journals, books, papers, web contents, have been made throughout this disclosure, including to the Supplementary. The Supplementary, and all other such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

### EXAMPLES

### Example 1: Automatic Vessel Segmentation for Medical Imaging Using the Standardized Difference of Means (SDM) Velocity

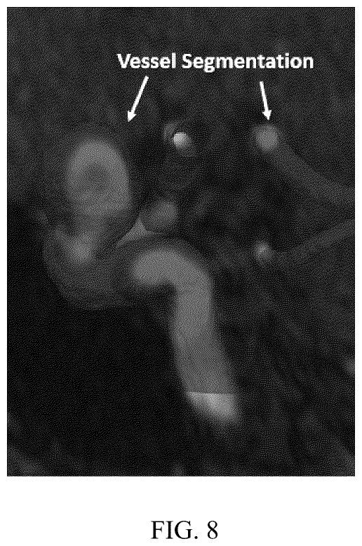

Vessel segmentation is the process of identifying regions in the medical image containing regions of blood or cerebral spinal fluid (CSF) flow. See FIG. 8. Accurate segmentation is critical for reliably assessing biomarkers of disease. For example, wall shear stress (WSS) is associated with atherogenesis and aneurysm initiation and growth. Poor vessel segmentation introduces error in assessing the location of the wall and, in turn, increases error in WSS estimates.

### Segmentation is Commonly Performed Manually

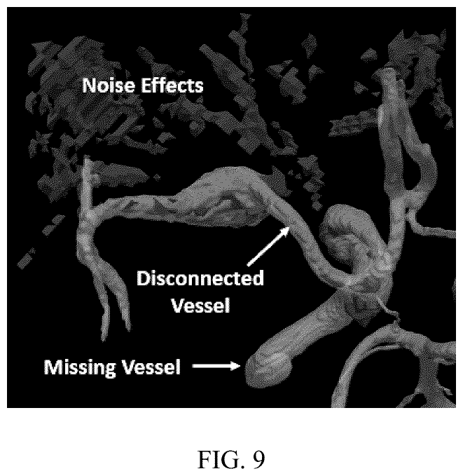

Manual segmentation is time-consuming, and the results can vary across users. See FIG. 9. Automatic segmentation algorithms are needed to increase the scale and accuracy of biomedical research. Many existing automatic segmentation methods are highly sensitive to error in medical images. Robust algorithms are needed to reliably study biomarkers of disease.

We introduce the standardized difference of means (SDM) velocity as a feature to automatically segmenting biological flow using time resolved velocity measurements. Thus far, we have applied the SDM method to 4D flow MRI measurements in the cerebral vasculature (see manuscript in preparation). The SDM segmentation method is generalizable across:
Biological flow types, e.g., CSF and blood flow;
Vascular territories, e.g., cerebral and abdominal vessels; and
Imaging modalities, e.g., MRI and ultrasound.

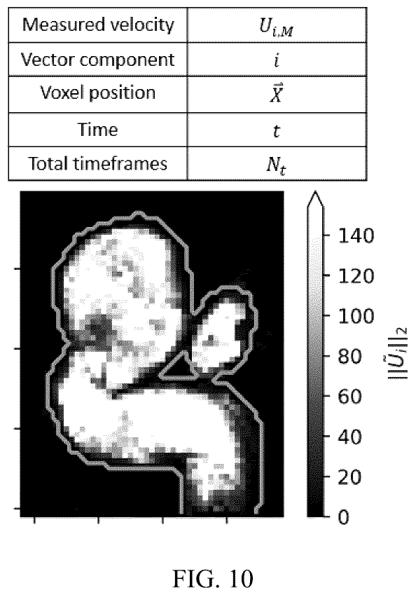

We discovered that a standardized velocity metric (SDM velocity) is particularly sensitive to voxels with fluid flow measured using 4D flow MRI. See FIG. 10. The SDM velocity, Ũᵢ, is defined as the difference between the time-averaged measured velocity at each voxel and the mean tissue velocity (\hat{μ}ᵢ) relative to the standard error,

$${stddev}_{t} [ {U}_{i , M} ( \vec{X} , t ) ] / \sqrt{{N}_{t}} : {\tilde{U}}_{i} ( \vec{X} ) = \frac{{mean}_{t} [ {U}_{i , M} ( \vec{X} , t ) ] - {\hat{μ}}_{i}}{{stddev}_{t} [ {U}_{i , M} ( \vec{X} , t ) ] / \sqrt{{N}_{t}}}$$

SDM segmentations are generated by identifying voxels with significant values of Ũᵢ compared to tissue. The figure to the right show a map of Ũᵢ magnitude. Voxels in the vessel have much higher values of SDM velocity magnitude.

We embed the SDM velocity in the SDM algorithm to automatically generate the segmentation (a.k.a. mask) and a map of p-values. See FIG. 1. The p-values quantify the significance of fluid flow effects and express the segmentation performance in all voxels. The SDM algorithm only operates on the measured velocity and two user-defined parameters: a blurring factor (ρ_B\hat{ }2) and the critical p-value (p_crit)

There are four steps in the current SDM algorithm:
Initialization: provides a crude approximation of tissue voxel locations;
Iteration: Iteratively refines the vessel segmentation based on significant values of the SDM velocity;
Isolation: Removes erroneous voxels from the converged segmentation; and
Dilation: Incorporates near-wall voxels into the SDM segmentation.

We validate the SDM segmentations against another automatic segmentation method, pseudo-complex difference (PCD), and high-resolution time-of-flight (TOF) angiography in the cerebral vasculature PCD segmentations are evaluated using both the signal magnitude and velocity from 4D flow MRI as defined by Schnell et al. See FIG. 6. TOF segmentations are clinically considered the vessel's “ground truth”. Qualitative results show the SDM segmentations closely represent TOF even in large aneurysms with very slow blood flow. The PCD segmentation misses many voxels in the vessel.

We assess how accurately the segmentations identify the vessel wall location. See FIGS. 4 and 7. We quantify this as the minimum distances from each actual surface voxel to the predicted vessel surface (d_min\hat{ }*). The SDM segmentations better assess the vessel wall than PCD segmentations across all in vitro phantom scales and in vivo patient measurements. In vitro and in vivo, the SDM segmentations are 49.6% and 71.9% closer to the actual vessel wall than the PCD segmentations as measured by the RMS distance, respectively Accurate wall segmentation will promote more accurate hemodynamic parameter assessment, e.g., WSS.

We quantify the performance of the segmentations in assessing all voxels. See Tables VIII and IX. We evaluate the sensitivity (Sen.), specificity (Spec.), precision (Prec.), negative predictive value (NPV), and balanced accuracy (Bal. Acc.). The SDM segmentations are more sensitive to voxels with flow than the PCD method in vitro and in vivo. The SDM method demonstrates an approximate 48% increase in sensitivity in vitro and 70% in vivo compared to the PCD approach. Accurate vessel segmentations will promote the assessment of accurate biomarkers associated with cardiovascular disease.

TABLE VIII
IN VITRO OVERLAP METRICS

| | |
|---|---|
| PCD | SDM |

| METRIC | 1-TO-1 | 2-TO-4 | 1-TO-1 | 2-TO-1 |
|---|---|---|---|---|
| Sen. | 49.77% | 49.24% | 97.67% | 97.32% |
| Spec. | 99.95% | 99.89% | 98.53% | 98.29% |
| Pre | 97.23% | 96.85% | 69.05% | 79.67% |
| NPV | 98.35% | 96.60% | 99.92% | 99.81% |
| Bal. Acc | 74.86% | 74.56% | 98.07% | 97.80% |

TABLE IX
IN VIVO OVERLAP METRICS FOR ALL PATIENTS

| METRIC | PCD | SDM |
|---|---|---|
| Sen. | 16.49% | 86.63% |
| Spec. | 99.71% | 94.73% |
| Bal. Acc | 58.10% | 90.68% |

The data herein show the SDM velocity as a feature to automatically segment biological flow using time resolved velocity measurements. We embedded the SDM velocity in an iterative algorithm to identify voxels with significant flow effects. The SDM algorithm serves as a robust method to segment vessels across a range of scan settings and imaging conditions. This consistent performance of the SDM method enables more accurate computation of hemodynamic and morphological metrics.

What is claimed is:

1. A method for performing vessel segmentation from flow data, the method comprising:
receiving flow data representative of flow in a vessel;
identifying net flow effects in the flow data according to a standardized difference of means (SDM) velocity that involves quantifying a ratio between net flow and observed flow pulsatility in each voxel of the received flow data; and
identifying voxels with higher SDM velocity values than stationary tissue voxels, thereby performing vessel segmentation from 4D MRI flow data.

2. The method of claim 1, wherein the method further comprises generating a P-value for each voxel to estimate segmentation accuracy.

3. The method of claim 1, wherein the SDM velocity, Ũᵢ, is defined as a difference between a time-averaged measured velocity at each voxel and a mean tissue velocity (ûᵢ) relative to a standard error
$${stddev}_{t} [ {U}_{i , M} ( \vec{X} , t ) ] / \sqrt{{N}_{t}} : {\tilde{U}}_{i} ( \vec{X} ) = \frac{{mean}_{t} [ {U}_{i , M} ( \vec{X} , t ) ] - {\hat{μ}}_{i}}{{stddev}_{t} [ {U}_{i , M} ( \vec{X} , t ) ] / \sqrt{{N}_{t}}} .$$

4. The method of claim 3, wherein SDM segmentations are generated by identifying voxels with significant values of Ũᵢ compared to tissue.

5. The method of claim 1, wherein the method further comprises initially providing an approximation of tissue voxel locations.

6. The method of claim 5, wherein the method further comprises iteratively refining the vessel segmentation based on significant values of the SDM velocity.

7. The method of claim 6, wherein the method further comprises removing erroneous voxels from converged segmentation.

8. The method of claim 7, wherein the method further comprises incorporating near-wall voxels into the SDM segmentation.

9. The method of claim 1, wherein the method further comprises using the vessel segmentation results to assess biomarkers of disease.

10. The method of claim 9, wherein the disease is cardiovascular disease.

11. A system for performing vessel segmentation from flow data, the system comprising a processor configured to:
receive flow data representative of flow in a vessel;
identify net flow effects in the flow data according to a standardized difference of means (SDM) velocity that involves quantifying a ratio between net flow and observed flow pulsatility in each voxel of the received flow data; and
identify voxels with higher SDM velocity values than stationary tissue voxels, thereby performing vessel segmentation from flow data.

12. The system of claim 11, wherein the method further comprises generating a P-value for each voxel to estimate segmentation accuracy.

13. The system of claim 11, wherein the SDM velocity, Ũᵢ, is defined as a difference between a time-averaged measured velocity at each voxel and a mean tissue velocity (ûᵢ) relative to a standard error,
$${stddev}_{t} [ {U}_{i , M} ( \vec{X} , t ) ] / \sqrt{{N}_{t}} : {\tilde{U}}_{i} ( \vec{X} ) = \frac{{mean}_{t} [ {U}_{i , M} ( \vec{X} , t ) ] - {\hat{μ}}_{i}}{{stddev}_{t} [ {U}_{i , M} ( \vec{X} , t ) ] / \sqrt{{N}_{t}}} .$$

14. The system of claim 13, wherein SDM segmentations are generated by identifying voxels with significant values of Ũᵢ compared to tissue.

15. The system of claim 11, wherein the method further comprises initially providing an approximation of tissue voxel locations.

16. The system of claim 15, wherein the method further comprises iteratively refining the vessel segmentation based on significant values of the SDM velocity.

17. The system of claim 16, wherein the method further comprises removing erroneous voxels from converged segmentation.

18. The system of claim 17, wherein the method further comprises incorporating near-wall voxels into the SDM segmentation.

19. The system of claim 11, wherein the method further comprises using the vessel segmentation results to assess biomarkers of disease.

20. The system of claim 19, wherein the disease is cardiovascular disease.
